(19) United States
(12) Patent Application Publication
(10) Pub. No.: US 2022/0114729 A1
(43) Pub. Date: Apr. 14, 2022

(54) X-RAY IMAGING APPARATUS, IMAGE PROCESSING METHOD, AND GENERATION METHOD OF TRAINED MODEL

(71) Applicant: SHIMADZU CORPORATION, Kyoto (JP)

(72) Inventors: Erzhong HU, Kyoto-shi, (JP); Naomasa HOSOMI, Kyoto-shi, (JP); Tomohiro NAKAYA, Kyoto-shi (JP)

(21) Appl. No.: 17/496,534

(22) Filed: Oct. 7, 2021

(30) Foreign Application Priority Data
Oct. 14, 2020 (JP) 2020-173580

(51) Int. Cl. G06T 7/00; G06T 5/50; G06T 11/00; G06T 7/11; G06K 9/62; A61B 6/12; A61B 6/00

(52) U.S. Cl. CPC G06T 7/0014; G06T 5/50; G06T 11/001; G06T 7/11; G06K 9/6256; G06K 2209/057; A61B 6/5211; A61B 6/463; G06T 2207/20081; G06T 2207/10116; G06T 2207/20224; A61B 6/12

(57) ABSTRACT

This X-ray imaging apparatus includes an X-ray irradiation unit, an X-ray detection unit, an X-ray image generation unit, and a control unit. The control unit includes: an enhanced image generation unit for generating an enhanced image in which a foreign object included in the X-ray image has been enhanced; an identification image generation unit for generating an identification image for identifying the foreign object by coloring a portion corresponding to the enhanced foreign object based on the enhanced image; and an image output unit for outputting an identification image.

### CROSS-REFERENCE TO RELATED APPLICATIONS

The priority application number JP2020-173580, entitled “X-Ray Imaging Apparatus, Image Processing Method, and Generation Method of Trained Model”, filed on Oct. 14, 2020, invented by HU Erzhong, Naomasa HOSOMI, and Tomohiro NAKAYA, upon which this patent application is based is hereby incorporated by reference.

### BACKGROUND OF THE INVENTION

### Field of the Invention

The present invention relates to an X-ray imaging apparatus, an image processing method, and a generation method of a trained model. In particular, the present invention relates to an X-ray imaging apparatus, an image processing method, and a generation method of a trained model, which are configured to perform X-ray imaging to identify a foreign object left behind in a body of a subject after a surgical operation.

### Description of the Background Art

Conventionally, a radiographic imaging system (X-ray imaging apparatus) has been known in which a confirmatory radiation test (X-ray imaging) is performed after a laparotomy surgical operation to confirm the presence or absence of a hemostatic gauze (foreign object). Such a radiographic imaging system is disclosed in, for example, Japanese Unexamined Patent Application Publication No. 2019-180605.

The radiographic imaging system described in Japanese Unexamined Patent Application Publication No. 2019-180605 performs processing of a radiographic image in accordance with the processing procedures set in advance according to the test purpose. For example, in a case where processing procedures for confirming the presence or absence of a hemostatic gauze after a laparotomy surgical operation are set in advance, foreign object enhancement processing is performed on the captured image. The radiographic imaging system described in Japanese Unexamined Patent Application Publication No. 2019-180605 displays an abdominal image in which foreign object enhancement processing has been performed so that a foreign object in a body of a subject can be easily recognized.

Here, although not specifically described in the above-described Japanese Unexamined Patent Application Publication No. 2019-180605, in the case of performing foreign object enhancement processing on a captured X-ray image in order for the operator, such as, e.g., a doctor, to confirm the presence or absence of a foreign object, such as, e.g., a hemostatic gauze (surgical operation gauze), left behind in a body of a subject (object) after a surgical operation, as in the radiographic imaging system of the above-described Japanese Unexamined Patent Application Publication No. 2019-180605, as the foreign object enhancement processing, it is conceivable to execute image processing, such as, e.g., edge detection processing, to generate an enhanced image.

However, in a case where the foreign object enhancement processing, such as, e.g., edge detection processing, is performed on the captured X-ray image, not only the contour of the foreign object but also contours of structures, such as, e.g., bones of the subject, are enhanced. With this, the visibility of the foreign object in the enhanced image deteriorates, which makes it difficult to confirm the foreign object included in the captured X-ray image.

### SUMMARY OF THE INVENTION

The present invention has been made to solve the aforementioned problems. It is an object of the present invention to provide an X-ray imaging apparatus, an image processing method, and a generation method of a trained model which are capable of easily identifying a foreign object included in an X-ray image when foreign object verification is performed by generating an enhanced image in which the foreign object left behind in a body of a subject is enhanced.

In order to attain the above-described object, the X-ray imaging apparatus according to the first aspect of the present invention is an X-ray imaging apparatus for performing X-ray imaging to identify a foreign object left behind in a body of a subject after a surgical operation, comprising:

an X-ray irradiation unit configured to irradiate the subject with X-rays;

an X-ray detection unit configured to detect X-rays emitted from the X-ray irradiation unit;

an X-ray image generation unit configured to generate an X-ray image based on a detection signal of X-rays detected by the X-ray detection unit; and

a control unit,

wherein the control unit includes:

an enhanced image generation unit configured to generate an enhanced image in which a foreign object included in an X-ray image has been enhanced, based on the X-ray image and a removed image in which the foreign object included in the X-ray image has been removed;

an identification image generation unit configured to generate an identification image for identifying the foreign object by coloring a portion corresponding to the enhanced foreign object, based on the enhanced image generated by the enhanced image generation unit; and

an image output unit configured to output the identification image generated by the identification image generation unit.

An image processing method according to a second aspect of the present invention includes the steps of:

irradiating a subject with X-rays to identify a foreign object left behind in a body of a subject after a surgical operation;

detecting the X-rays;

generating an X-ray image based on a detection signal of the detected X-rays;

generating an enhanced image in which the foreign object included in the X-ray image has been enhanced, based on the X-ray image and a removed image in which the foreign object included in the X-ray image has been removed from the X-ray image by a trained model generated by machine learning;

generating an identification image for identifying the foreign object by coloring a portion corresponding to the enhanced foreign object, based on the generated enhanced image; and outputting the generated identification image.

The generation method of a trained model according to a third aspect of the present invention includes the steps of:

acquiring a training input X-ray image generated to simulate an X-ray image generated by irradiating a subject after a surgical operation in which a foreign object is left behind in the body with X-rays;

acquiring a training output removed image in which the foreign object has been removed from the training input X-ray image; and

generating a trained model for outputting a removed image in which the foreign object included in the X-ray image has been removed from the X-ray image by machine learning, based on the training input X-ray image and the training output removed image, in order to generate an identification image for identifying the foreign object by coloring a portion corresponding to the foreign object.

In the X-ray imaging apparatus according to the first aspect of the present invention and the image processing method according to the second aspect of the present invention, an enhanced image in which a foreign object included in an X-ray image has been enhanced is generated based on the X-ray image and a removed image in which the foreign object included in the X-ray image has been removed from the X-ray image. An identification image for identifying the foreign object is generated by coloring the portion corresponding to the enhanced foreign object based on the generated enhanced image.

With this, unlike the case in which in the enhanced image, both the structure of a bone or the like of a subject and a foreign object included in the subject are enhanced by foreign object enhancement processing, such as, e.g., edge detection processing, by extracting the foreign object based on the X-ray image containing the foreign object and the removed image in which the foreign object has been removed, it is possible to enhance the foreign object without enhancing the structure of the bone or the like of the subject. Therefore, in the case of generating an identification image for identifying the foreign object based on the enhanced image in which the foreign object is enhanced, the foreign object in the identification image can be effectively extracted and colored. Therefore, the colored foreign object can be easily identified by visually recognizing the identification image in which the portion corresponding to the enhanced foreign object has been effectively colored. As a result, the foreign object included in the X-ray image can be easily identified when confirming the foreign object by generating the enhanced image in which the foreign object left behind in the body of the subject has been enhanced.

In a generation method of a trained model according to the third aspect, in order to generate an identification image for identifying the foreign object by coloring the portion corresponding to the foreign object, a trained model for outputting a removed image in which the foreign object included in the X-ray image has been removed is generated based on the training input X-ray image and a training output removed image.

With this, it possible to generate a removed image in which the foreign object has been removed based on the trained model generated by machine learning, and therefore the foreign object in the X-ray image can be extracted based on the X-ray image including the foreign object and the removed image in which the foreign object has been removed. Thus, an enhanced image in which the foreign object has been enhanced while suppressing the enhancement to the structure of a bone or the like of a subject can be generated. Therefore, by coloring the portion corresponding to the enhanced foreign object, an identification image for identifying the foreign object can be easily generated. As a result, the portion corresponding to the colored foreign object can be easily identified by visually recognizing the identification image, and therefore, it is possible to provide a generation method of a trained model capable of easily identifying the foreign object included in the X-ray image.

### BRIEF DESCRIPTION OF THE DRAWINGS

FIG. 1 is a diagram for explaining a configuration of an X-ray imaging apparatus according to one embodiment of the present invention.

FIG. 2 is a block diagram for explaining a configuration of the X-ray imaging apparatus according to one embodiment.

FIG. 3 is a diagram showing an example of an X-ray image of a subject in which a foreign object is left behind in a body according to one embodiment.

FIG. 4 is a diagram for explaining a generation of a removed image according to one embodiment.

FIG. 5 is a diagram for explaining a division of an X-ray image by a removed image generation unit according to one embodiment.

FIG. 6 is a diagram for explaining a generation of a trained model according to one embodiment.

FIG. 7 is a diagram for explaining a generation of an enhanced image according to one embodiment.

FIG. 8 is a diagram for explaining a generation of an identification image according to one embodiment.

FIG. 9 is a diagram for explaining a display of a display unit according to one embodiment.

FIG. 10 is a flowchart for explaining a generation method of a trained model according to one embodiment.

FIG. 11 is a flowchart for explaining an image processing method according to one embodiment.

### DESCRIPTION OF THE PREFERRED EMBODIMENT

Hereinafter, some embodiments in which the present invention is embodied will be described with reference to the attached drawings.

### (Overall Configuration of X-Ray Imaging Apparatus)

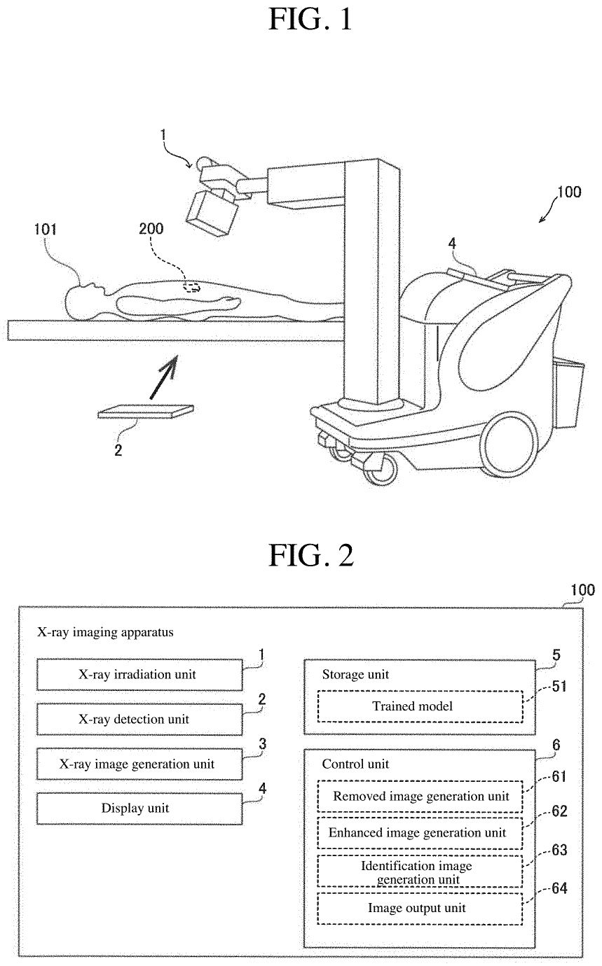

With reference to FIG. 1 to FIG. 9, an X-ray imaging apparatus 100 according to an embodiment of the present invention will be described.

As shown in FIG. 1, the X-ray imaging apparatus 100 performs X-ray imaging to identify a foreign object 200 left behind in a body of a subject 101 after a surgical operation. The X-ray imaging apparatus 100 performs X-ray imaging for confirming whether or not the foreign object 200 is left behind in the body with respect to the subject 101 to which a laparotomy surgical operation has been performed in a surgical operation room. The X-ray imaging apparatus 100 is, for example, an X-ray imaging apparatus for rounds capable of moving the entire apparatus. The foreign object 200 includes, for example, a surgical operation gauze, a suture needle, and a forceps (e.g., a hemostatic forceps).

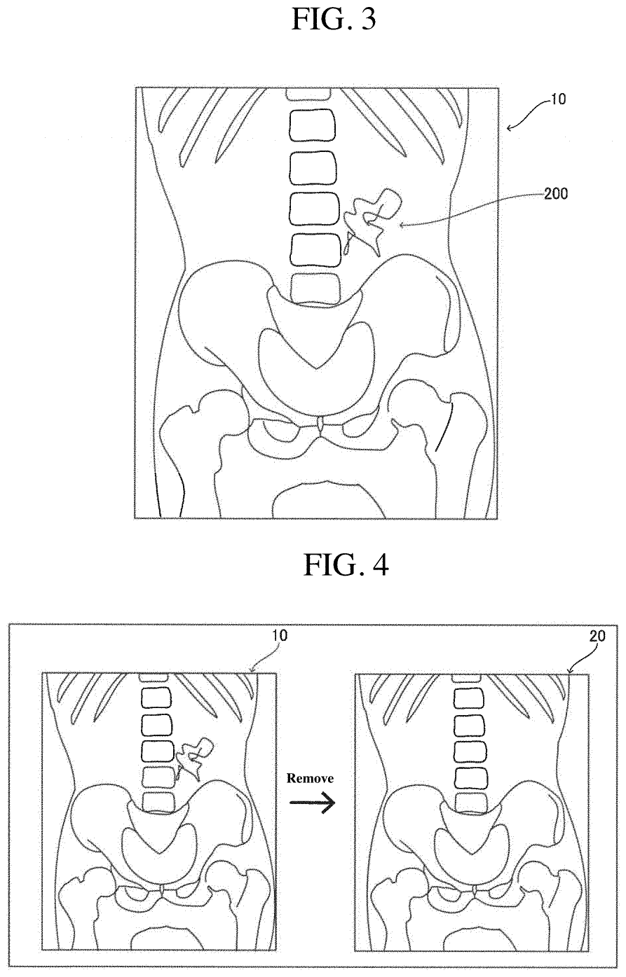

Generally, when a surgical operation, such as, e.g., an open surgical operation, has been performed, an operator, such as, e.g., a doctor, performs confirmatory X-ray imaging with respect to the subject 101 so that a foreign object 200, such as, e.g., a surgical operation gauze, a suture needle, and a forceps, is not left behind in the subject 101 after the closure. An operator, such as, e.g., a doctor, confirms that no foreign object 200 has been left behind in the body of the subject 101 by visually checking the captured X-ray image 10 (see FIG. 3).

### <X-Ray Imaging Apparatus>

As shown in FIG. 2, the X-ray imaging apparatus 100 is provided with an X-ray irradiation unit 1, an X-ray detection unit 2, an X-ray image generation unit 3, a display unit 4, a storage unit 5, and a control unit 6.

The X-ray irradiation unit 1 irradiates the subject 101 after a surgical operation with X-rays. The X-ray irradiation unit 1 includes an X-ray tube that emits X-rays when a voltage is applied.

The X-ray detection unit 2 detects the X-rays transmitted through the subject 101. The X-ray detection unit 2 outputs a detection signal based on the detected X-rays. The X-ray detection unit 2 includes, for example, an FPD (flat panel detector). Further, the X-ray detection unit 2 is configured as a wireless-type X-ray detector and outputs a detection signal as a radio signal. More specifically, the X-ray detection unit 2 is configured to be able to communicate with the X-ray image generation unit 3, which will be described later, by a wireless connection via a wireless LAN or the like, and outputs a detection signal as a wireless signal to the X-ray image generation unit 3.

As shown in FIG. 3, the X-ray image generation unit 3 controls X-ray imaging by controlling the X-ray irradiation unit 1 and the X-ray detection unit 2. The X-ray image generation unit 3 generates an X-ray image 10 based on the detection signal of the X-rays detected by the X-ray detection unit 2. The X-ray image generation unit 3 is configured to be able to communicate with the X-ray detection unit 2 by a wireless connection via a wireless LAN or the like. The X-ray image generation unit 3 includes a processor, such as, e.g., an FPGA (Field-Programmable Gate Array). The X-ray image generation unit 3 outputs the generated X-ray image 10 to the control unit 6 to be described later.

The X-ray image 10 is an image acquired by X-ray imaging the abdomen of the subject 101 after a surgical operation. For example, the X-ray image 10 includes a surgical operation gauze as the foreign object 200. Note that the surgical operation gauze is woven with a contrast yarn which is hard to transmit X-rays so as to be visible in the X-ray image 10 by X-ray imaging after a surgical operation.

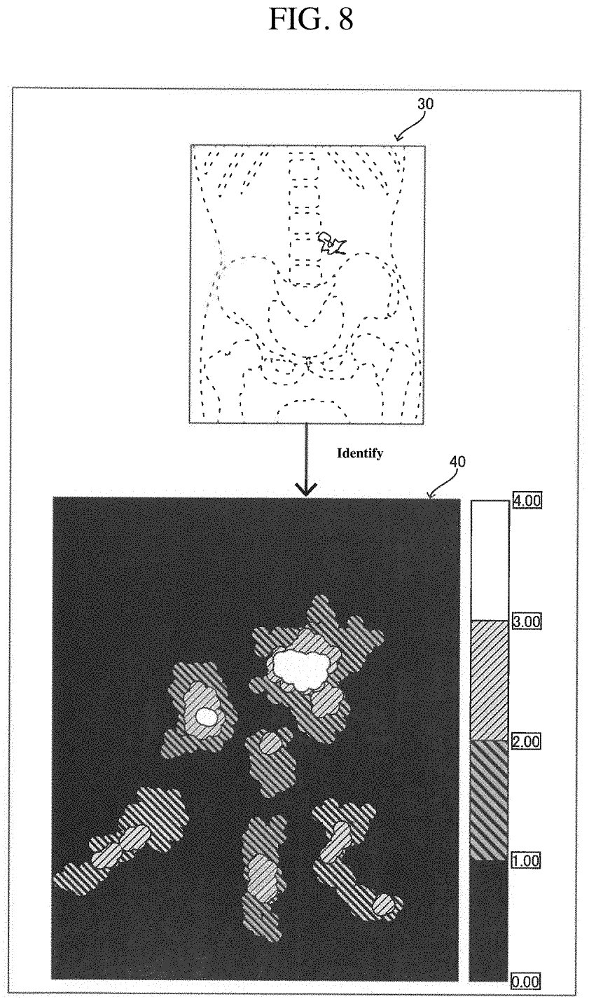

The display unit 4 includes, for example, a touch panel type liquid crystal display. The display unit 4 displays the captured X-ray image 10. The display unit 4 displays an identification image 40 (see FIG. 8) output by an image output unit 64 to be described later. Further, the display unit 4 is configured to accept an input operation for operating the X-ray imaging apparatus 100 by an operator, such as, e.g., a doctor, based on a manipulation of the touch panel.

The storage unit 5 is configured by a storage device, such as, e.g., a hard disk drive. The storage unit 5 stores the image data, such as, e.g., the X-ray image 10 generated by the X-ray image generation unit 3 and the identification image 40 (see FIG. 8) generated by the control unit 6 to be described later. The storage unit 5 is configured to store various set values for operating the X-ray imaging apparatus 100. Further, the storage unit 5 stores programs to be used for processing the control of the X-ray imaging apparatus 100 by the control unit 6. The storage unit 5 stores a trained model 51, which will be described later.

The control unit 6 is a computer configured to include, for example, a CPU (Central Processing Unit), a GPU (Graphics Processing Unit), a ROM (Read Only Memory), and a RAM (Random Access Memory). The control unit 6 includes, as functional components, a removed image generation unit 61, an enhanced image generation unit 62, an identification image generation unit 63, and an image output unit 64.

That is, the control unit 6 functions as the removed image generation unit 61, the enhanced image generation unit 62, the identification image generation unit 63, and the image output unit 64, by executing a predetermined control program. The removed image generation unit 61, the enhanced image generation unit 62, the identification image generation unit 63, and the image output unit 64 are functional blocks as software in the control unit 6, and are configured to function based on the command signal of the control unit 6 as hardware.

### (Generation of Removed Image)

As shown in FIG. 4, in this embodiment, the removed image generation unit 61 (control unit 6) generates the removed image 20 in which the foreign object 200 included in the X-ray image 10 has been removed from the X-ray image 10, based on the trained model 51 generated by machine learning.

The trained model 51 is generated by machine learning using deep learning. The trained model 51 is generated based on an U-Net, which is one type of a fully convolution network (FCN), for example. The trained model 51 is generated by learning to perform an image transformation (image reconstruction) for removing the portion estimated to be the foreign object 200 from the X-ray image 10 by transforming the pixel estimated to be the foreign object 200 from each pixel of the X-ray image 10.

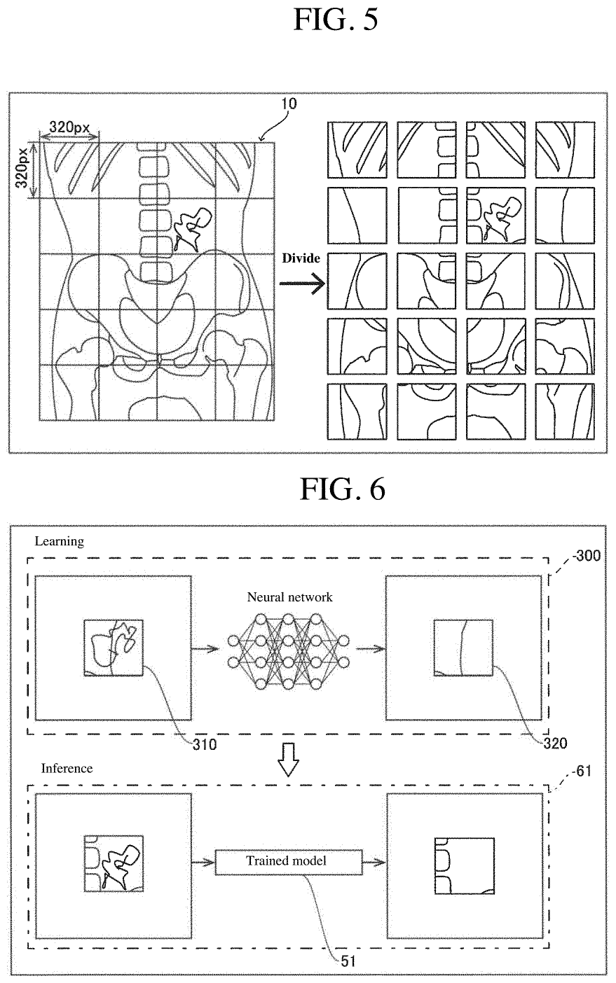

As shown in FIG. 5, in this embodiment, the removed image generation unit 61 (control unit 6) is configured to divide the X-ray image 10 into a plurality of regions and generate a removed image 20 from the X-ray image 10 divided into the plurality of regions, based on the trained model 51 generated by machine learning. Specifically, the removed image generation unit 61 cuts the X-ray image 10 into a size corresponding to the foreign object 200 included in the X-ray image 10.

For example, in a case where the foreign object 200 is a surgical operation gauze, the removed image generation unit 61 cuts the X-ray image 10 such that one cut X-ray image 10 has a size slightly larger than the typical size of the foreign object 200 (surgical operation gauze) included in the X-ray image 10. Specifically, the removed image generation unit 61 down-samples (resizes) the X-ray image 10 generated by the X-ray image generation unit 3 and then divides it into five equal regions vertically and four equal regions horizontally, thereby dividing it into twenty square regions with one side of 320 pixels. Then, the removed image generation unit 61 executes processing for removing the foreign object 200 based on the trained model 51 generated by machine learning for each region of the cut square. The removed image generation unit 61 generates the removed image 20 in which the foreign object 200 has been removed from the X-ray image 10 by composing the region of the 20 squares from which the foreign object 200 has been removed so as to correspond to the pre-cut X-ray image 10 after executing the processing of removing the foreign object 200 for each of the 20 square regions.

It should be noted that the X-ray image 10 by the removed image generation unit 61 may be cut into rectangles instead of squares. Further, the X-ray image 10 may be cut into a predetermined size by the removed image generation unit 61 regardless of the size of the foreign object 200.

### (Generation of Trained Model)

As shown in FIG. 6, in this embodiment, the trained model 51 is generated by machine learning so as to remove the foreign object 200 including a surgical operation gauze, a suture needle, and a forceps from the X-ray image 10. The trained model 51 is pre-generated by a learning device 300 separated from the X-ray imaging apparatus 100. The learning device 300 is a computer configured to include, for example, a CPU, a GPU, a ROM, and a RAM. The trained model 51 is generated by machine learning using deep learning, based on a training input X-ray image 310 and a training output removed image 320.

The training input X-ray image 310 is generated by the learning device 300 so as to simulate the X-ray image 10 reflecting the subject 101 with the foreign object 200 left behind in the body. The learning device 300 acquires, for example, a simulated X-ray image generated by irradiating a human body phantom simulating a human body with X-rays and a foreign object image generated by X-ray imaging the foreign object 200, such as, e.g., a medical gauze, a suture needle, and a forceps.

Then, the learning device 300 cuts the acquired simulated X-ray image in order to generate a training input X-ray image 310, similarly to the cutting of the X-ray image 10 by the removed image generation unit 61 of the control unit 6 of the X-ray imaging apparatus 100. For example, in the case where the removed image generation unit 61 cuts the X-ray image 10 into 320 pixel squares, the learning device 300 likewise cuts the simulated X-ray image into 320 pixel squares. Then, the learning device 300 generates the training input X-ray image 310 by composing the cut simulated X-ray image and the foreign object image. The learning device 300 generates a plurality of training input X-ray image 310 by changing parameters (angle, density, size, etc.) for combining the simulated X-ray image and the foreign object image to several conditions.

The training input X-ray image 310 used as an input in machine learning for generating the trained model 51 is generated by the learning device 300 so as to have a condition (size) similar to that of the X-ray image 10 used as an input in the inference using the trained model 51. The training input X-ray image 310 may include a simulated X-ray image (with no foreign object image composed) that does not include the foreign object 200. Alternatively, instead of the simulated X-ray image, the training input X-ray image 310 may be generated using an image that is an actual X-ray image of a human body.

The training output removed image 320 is an image in which the foreign object 200 has been removed from the plurality of generated training input X-ray images 310. For example, the learning device 300 acquires, as the training output removed image 320, a simulated X-ray image before the foreign object image used to generate the training input X-ray image 310 is composed. In other words, the training output removed image 320 is acquired as a segmented image, similarly to the training input X-ray image 310.

The learning device 300 receives the training input X-ray image 310, outputs the training output removed image 320, and learns by machine learning to generate the trained model 51. That is, the learning device 300 generates the trained model 51 by machine learning using the training input X-ray image 310 and the training output removed image 320 as training data (training set). The learning device 300 generates the trained model 51 by learning using a plurality of training input X-ray images 310 and a plurality of training output removed images 320.

The generated trained model 51 is stored in advance in the storage unit 5 of the X-ray imaging apparatus 100 before performing X-ray imaging. The trained model 51 may be stored in the storage unit 5 of the X-ray imaging apparatus 100 from the learning device 300 via a network, or may be stored in the storage unit 5 via a storage medium, such as, e.g., a flash memory.

### (Generation of Enhanced Image)

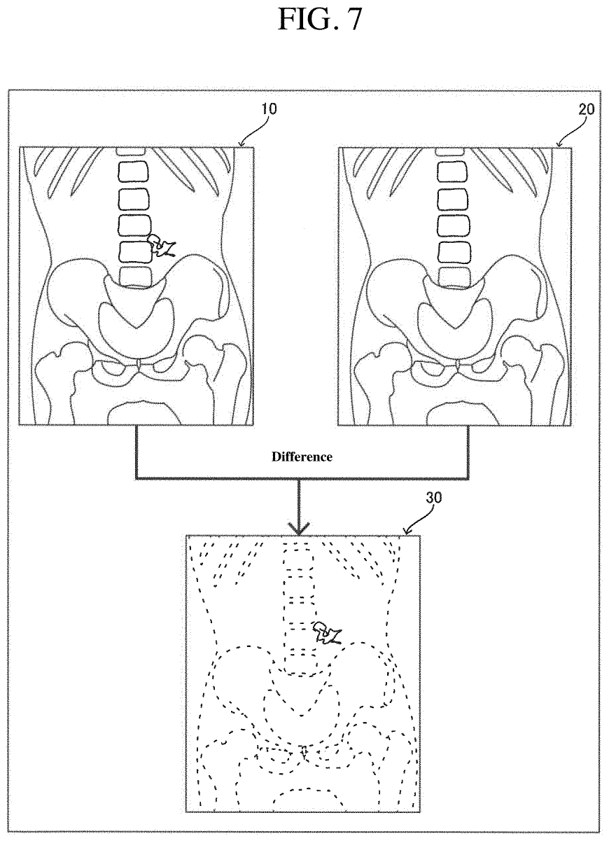

As shown in FIG. 7, in this embodiment, the enhanced image generation unit 62 (control unit 6) generates an enhanced image 30 in which the foreign object 200 included in the X-ray image 10 has been enhanced, based on the X-ray image 10 and the removed image 20 in which the foreign object 200 included in the X-ray image 10 has been removed by the trained model 51 generated by machine learning. Specifically, the enhanced image generation unit 62 is configured to generate the enhanced image 30 in which the foreign object 200 included in the X-ray image 10 has been enhanced by acquiring the difference between the X-ray image 10 and the removed image 20.

In particular, the enhanced image generation unit 62 generates the enhanced image 30 by subtracting the removed image 20 from the X-ray image 10. That is, the enhanced image generation unit 62 generates the enhanced image 30 by subtracting each pixel value of the corresponding pixel of the removed image 20 from each pixel value of the pixel included in the X-ray image 10. The enhanced image 30 is an image in which the foreign object 200 included in the X-ray image 10 has been enhanced and the enhancement of structures other than the foreign object 200 (e.g., the bones of the subject 101) in the X-ray image 10 has been suppressed.

### (Generation of Identification Image)

As shown in FIG. 8, in this embodiment, the identification image generation unit 63 (control unit 6) generates an identification image 40 for identifying the foreign object 200 by coloring the portion corresponding to the enhanced foreign object 200, based on the enhanced image 30 generated by the enhanced image generation unit 62 (control unit 6).

Specifically, in this embodiment, the identification image generation unit 63 (control unit 6) is configured to generate the identification image 40 by identifying the linear structure (shape) of the foreign object 200 from the enhanced image 30 and coloring based on the identified linear structure. In particular, the identification image generation unit 63 is configured to acquire the density in a predetermined region including the site in which the linear structure of the identified foreign object 200 is located from the enhanced image 30, and generate the identification image 40 as a heat map image (color map image) colored so as to change according to the density.

For example, the identification image generation unit 63 performs binary processing on the generated enhanced image 30. Then, the identification image generation unit 63 detects the density of the linear structure in the enhanced image 30 subjected to the binary processing, thereby identifying the portion (pixel) in which the linear structure is included. In particular, the identification image generation unit 63 performs the pattern recognition on the binarized enhanced image 30 by extracting the feature quantity from the enhanced image 30 to identify the linear structure.

For example, the identification image generation unit 63 extracts higher-order local autocorrelation (HLAC: Higher-order Local AutoCorrelation) features as the feature quantity. The identification image generation unit 63 acquires, for example, one pixel of the enhanced image 30 as a reference point. The identification image generation unit 63 extracts the feature quantity according to the local autocorrelation feature of a predetermined region (centered) including a reference point. Then, the identification image generation unit 63 measures the degree of coincidence between the extracted feature quantity and the feature quantity of the preset linear structure, thereby identifying (detecting) the linear structure in a predetermined region including the reference point. The identification image generation unit 63 acquires the detected value of the linear structure in a predetermined region including the reference point as the density at the predetermined region of the linear structure at the reference point. The identification image generation unit 63 acquires the density (detected value) of the linear structure at each pixel of the enhanced image 30 by acquiring the local autocorrelation feature quantity using each pixel of the enhanced image 30 as a reference point. Here, the size of the predetermined region including the reference point may be, for example, a region of 3×3 pixels or a region of 9×9 pixels or the like larger than 3×3 pixels.

Then, the identification image generation unit 63 generates the identification image 40 by coloring each pixel, based on the linear structure density (detected value) acquired every pixel of the enhanced image 30. The identification image 40 is colored at each pixel such that, for example, based on red as a reference, the higher the value of the corresponding density is, the higher the brightness is, and the lower the value of the density is, the lower the brightness is. That is, the identification image 40 is generated such that the color becomes lighter and whiter as the value becomes larger, and the color becomes darker and blacker as the value becomes smaller. For example, the brightness of the color in the identification image 40 is set by values of 0 to 256 levels from 0 to 255. The identification image generation unit 63 sets the colors in the identification image 40 in such a manner that the range of the value of 0.0 or more and 4.0 or less of the density (detected value) of the acquired linear structure is associated with the brightness of 256 levels.

Note that when the density (detected value) is larger than 4.0, the same color as 4.0 (the color with the largest brightness value) is set. In FIG. 8, the difference in color is represented by the difference in hatching. In FIG. 8, the identification image 40 is shown such that the color (hatching) changes in a stepwise manner every 1.0 of the value of density (detected value). However, the identification image 40 may be generated such that the color gradually changes rather than that the color changes in a stepwise (discrete) manner.

The identification image generation unit 63 generates an identifiable identification image 40 capable of identifying how much the feature quantity in each pixel (each region) coincides with the feature quantity of the linear structure corresponding to the foreign object 200, by the color displayed as described above. Note that the color change in the identification image 40 may be represented by a color change (discrete color change) of about 4 or 5 levels by providing a threshold value, rather than a continuous color (brightness value) change of 256 levels.

Further, the identification (extraction) of the linear structure (shape) when generating the identification image 40 may be performed by acquiring the feature quantity for each predetermined region by pattern recognition using an extraction method of the feature quantity other than the higher-order local autocorrelation features, and by identifying (detecting) the density (degree of the pattern) of the linear structure (shape) and coloring it. Further, in the above description, an example is shown in which the surgical operation gauze is identified as the foreign object 200 has been described, but even in a case where the foreign object 200 is a suture needle, a forceps, or the like, the identification image 40 is similarly generated by identifying the linear structure (shape) from the enhanced image 30.

### (Display on Display Unit)

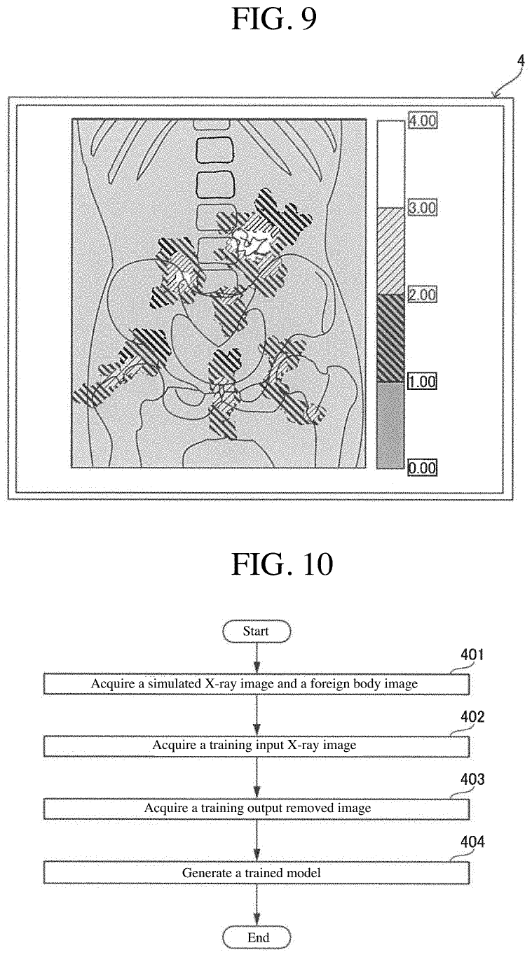

As shown in FIG. 9, in this embodiment, the image output unit 64 (control unit 6) outputs the identification image 40 generated by the identification image generation unit 63. Specifically, the image output unit 64 is configured to display the X-ray image 10 and the identification image 40 on the display unit 4. In this embodiment, the image output unit 64 is configured such that the X-ray image is displayed on the display unit 4 with the identification image 40 superimposed thereon.

The image output unit 64 performs image processing for making, for example, the transmittance of the identification image 40 to be 50%. Then, the image output unit 64 displays the identification image 40 with the 50% transmittance on the display unit 4 by superimposing it on the X-ray image 10. That is, the image output unit 64 causes the display unit 4 to display the X-ray image 10 with the identification image 40 subjected to the transmission processing superimposed on the X-ray image 10. The image output unit 64 causes the display unit 4 to display the X-ray image 10 with the identification image 40 superimposed thereon so that the foreign object 200 in the X-ray image 10 can be visually recognized in a colored state.

Note that it may be configured such that the image output unit 64 can switch between displaying the X-ray image 10 with the identification image 40 superimposed thereon and displaying only the X-ray image 10 without displaying the identification image 40, based on the input operation to the touch panel of the display unit 4. Further, it may be configured such that the image output unit 64 causes the display unit 4 to display the enhanced image 30 in addition to the identification image 40 and the X-ray image 10.

(Generation Method of Trained Model According to this Embodiment)

Next, with reference to FIG. 10, a generation method of a trained model according to this embodiment will be described. Note that the generation method of the trained model is performed by the learning device 300.

First, in Step 401, a simulated X-ray image of a human body phantom acquired by X-ray imaging and a foreign object image of the foreign object 200 acquired by X-ray imaging are acquired.

Next, in Step 402, a training input X-ray image 310 generated so as to simulate the X-ray image 10 generated by irradiating the subject 101 after a surgical operation in which the foreign object 200 is left behind in the body with X-rays is acquired. Specifically, the simulated X-ray image is cut into a plurality of images. Then, a training input X-ray image 310 generated by composing the cut simulated X-ray images and the plurality of foreign object images while changing the parameters (concentration, angle, size, and the like) is acquired.

Next, in Step 403, a training output removed image 320 in which the foreign object 200 has been removed from the training input X-ray image 310 is acquired. Specifically, the plurality of simulated X-ray images cut in Step 402 is acquired as the training output removed image 320 so as to correspond to the training input X-ray image 310.

Next, in Step 404, in order to generate the identification image 40 for identifying the foreign object 200 by coloring the portion corresponding to the foreign object 200, a trained model 51 for outputting the removed image 20 in which the foreign object 200 included in the X-ray image 10 has been removed from the X-ray image 10 by machine learning based on the training input X-ray image 310 and the training output removed image 320. More specifically, learning is performed by machine learning using deep learning with the training input X-ray image 310 as an input and the training output removed image 320 as an output, and a trained model 51 for generating the removed image 20 is generated.

(Image Processing Method by this Embodiment)

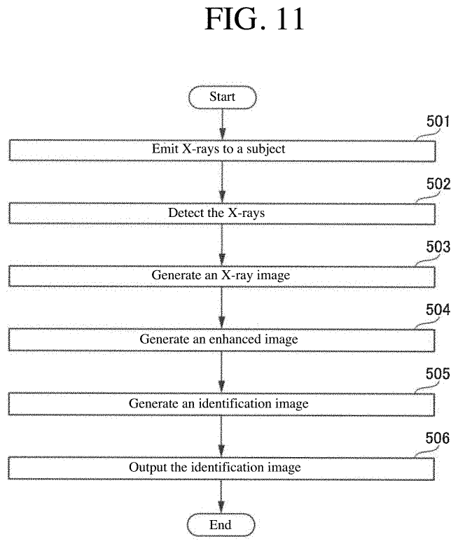

Next, with reference to FIG. 11, the control processing flow relating to the image processing method according to this embodiment will be described. Step 501 to Step 503 indicate control processing by the X-ray image generation unit 3, and Step 504 to Step 506 indicate control processing by the control unit 6.

First, in Step 501, the subject 101 is irradiated with X-rays to identify the foreign object 200 left behind in the body of the subject 101 after a surgical operation. Next, in Step 502, the emitted X-rays are detected. Next, in Step 503, an X-ray image 10 is generated based on the detection signal of the detected X-rays.

Next, in Step 504, an enhanced image 30 in which the foreign object 200 included in the X-ray image 10 is enhanced is generated, based on the X-ray image 10 and the removed image 20 in which the foreign object 200 included in the X-ray image 10 has been removed from the X-ray image 10 by the trained model 51 generated by machine learning. Specifically, the X-ray image 10 is divided into a plurality of regions, and a removed image 20 in which the foreign object 200 has been removed is generated from the X-ray image 10 divided into a plurality of regions based on the trained model 51. Then, by acquiring the difference between the X-ray image 10 and the removed image 20, an enhanced image 30 in which the foreign object 200 has been enhanced is generated.

Next, in Step 505, an identification image 40 is generated for identifying the foreign object 200 by coloring the portion corresponding to the enhanced foreign object 200 based on the generated enhanced image 30. Specifically, an identification image 40 is generated by identifying (detecting) the linear structure (shape) of the foreign object 200 from the enhanced image 30 and coloring based on the density (detection value) in a predetermined region including a site in which the identified linear structure is arranged.

Next, in Step 506, the generated identification image 40 is output. Specifically, the identification image 40 is superimposed on the X-ray image 10 and displayed on the display unit 4.

[Effects of this Embodiment]

In this embodiment, the following effects can be obtained.

As described above, the X-ray imaging apparatus 100 of this embodiment generates the X-ray image 30 in which the foreign object 200 included in the X-ray image 10 is enhanced, based on the X-ray image 10 and the removed image 20 in which the foreign object 200 included in the X-ray image 10 has been removed from the X-ray image 10 by the trained model 51 generated by machine learning. An identification image 40 for identifying the foreign object 200 is generated by coloring the portion corresponding to the enhanced foreign object 200 based on the generated enhanced image 30.

With this, unlike the case in which both the structure of the bone of the subject and the foreign object 200 included in the subject 101 enhanced by the foreign object enhancement processing, such as, e.g., edge detection processing, are enhanced, it is possible to emphasize the foreign object 200 without enhancing the structure of the bone or the like of the subject 101 by extracting the foreign object 200 based on the X-ray image 10 reflecting the foreign object 200 and the removed image 20 in which the foreign object 200 has been removed. For this reason, in the case of generating the identification image 40 for identifying the foreign object 200 based on the enhanced image 30 in which the foreign object 200 has been enhanced, the foreign object 200 in the identification image 40 can be effectively extracted and colored. Therefore, the colored foreign object 200 can be easily identified by visually recognizing the identification image 40 in which the portion corresponding to the enhanced foreign object 200 is effectively colored. As a result, in the case of generating the enhanced image 30 in which the foreign object 200 left behind in the body of the subject 101 has been enhanced, the foreign object 200 included in the X-ray image 10 can be easily identified.

Further, in the above-described embodiment, the following effects can be further obtained by the following configuration.

That is, in this embodiment, as described above, the identification image generation unit 63 (control unit 6) is configured to generate the identification image 40 by identifying the linear structure of the foreign object 200 from the enhanced image 30 and coloring based on the identified linear structure.

With this configuration, since the foreign object 200 included in the enhanced image 30 has a linear structure, it is possible to easily identify the portion corresponding to the foreign object 200 by identifying the linear structure from the enhanced image 30. Therefore, the identification image 40 in which the portion corresponding to the foreign object 200 is colored can be easily generated, and therefore, the foreign object 200 included in the X-ray image 10 can be easily identified by referring to the generated identification image 40.

In this embodiment, as described above, the identification image generation unit 63 (control unit 6) is configured to acquire the density in a predetermined region including the site in which the linear structure of the identified foreign object 200 is arranged from the enhanced image 30 and generate the identification image 40 as a heat map image colored so as to change according to the density.

With this configuration, the identification image 40 is colored according to the density in the predetermined region including the site in which the linear structure of the foreign object 200 is arranged, and therefore, it is possible to generate the identification image 40 in which the portion with higher density is enhanced. Therefore, in the enhanced image 30 generated so as to enhance the foreign object 200, since the density of the linear structure is high in the portion corresponding to the foreign object 200, the portion corresponding to the foreign object 200 in the identification image 40 can be more enhanced and colored. As a result, the foreign object 200 included in the X-ray image 10 can be more easily identified by visually recognizing the identification image 40.

In this embodiment, as described above, the control unit 6 further includes the removed image generation unit 61 for generating the removed image 20 in which the foreign object 200 included in the X-ray image 10 has been removed from the X-ray image 10, based on the trained model 51 generated by machine learning. The removed image generation unit 61 (control unit 6) is configured to divide the X-ray image 10 into a plurality of regions and generate the removed image 20 from the X-ray image 10 divided into a plurality of regions based on the trained model 51 generated by the machine learning.

The identification image generation unit 63 (control unit 6) is configured to generate the identification image 40 by identifying the linear structure of the foreign object 200 and coloring based on the identified linear structure from the enhanced image 30 generated based on the X-ray image 10 and the removed image 20 generated from the X-ray image 10 divided into a plurality of regions.

With this configuration, since the processing for removing the foreign object 200 using the trained model 51 by using the divided X-ray image as the input, the ratio of the foreign object 200 to the entire image of the input can be increased as compared with the case in which processing for removing the foreign object 200 using one X-ray image 10 as the input without dividing it is performed. Therefore, the accuracy of removing the foreign object 200 can be increased by inputting the divided X-ray image 10. As a result, since the foreign object 200 can be enhanced more accurately in the enhanced image 30, an operator, such as, e.g., a doctor, can more effectively identify the foreign object 200 by generating the removed image 20 from the X-ray image 10 which is divided into a plurality of regions.

In this embodiment, as described above, the enhanced image generation unit 62 (control unit 6) is configured to generate the enhanced image 30 in which the foreign object 200 included in the X-ray image 10 has been enhanced by acquiring the difference between the X-ray image 10 and the removed image 20. The identification image generation unit 63 (control unit 6) is configured to generate the identification image 40 by identifying the linear structure of the foreign object 200 from the enhanced image 30 generated by acquiring the difference between the removed image 20 and the X-ray image 10 and coloring based on the identified linear structure.

With this configuration, by acquiring the difference between the X-ray image 10 including the foreign object 200 and the removed image 20 not including the foreign object 200, it is possible to effectively generate the enhanced image 30 in which the foreign object 200 has been enhanced (extracted) because the edge of the structure, such as, e.g., a bone, has been removed. Therefore, in the case of identifying the linear structure of the foreign object 200 from the enhanced image 30, the portion corresponding to the foreign object 200 can be identified with high accuracy. As a result, the portion corresponding to the foreign object 200 can be colored with high accuracy, and therefore, the identification image 40 can be generated such that the portion corresponding to the foreign object 200 can be more effectively identified.

Further, in this embodiment, as described above, the foreign object 200 includes a surgical operation gauze, a suture needle, and a forceps. The identification image generation unit 63 (control unit 6) is configured to generate the identification image 40 by identifying the linear structure corresponding to the surgical operation gauze, the suture needle, or the forceps from the enhanced image 30 generated by the enhanced image generation unit 62 (control unit 6) and coloring based on the identified linear structure.

With this configuration, the surgical operation gauze, the suture needle, or the forceps, which is likely to be left behind in the body of the subject 101 after a surgical operation, can be colored so as to be easily identifiable in the identification image 40. As a result, an operator, such as, e.g., a doctor, can effectively identify the surgical operation gauze, the suture needle, or the forceps, which is likely to be left behind in the body of the subject 101 after a surgical operation, by visually recognizing the identification image 40.

Further, in this embodiment, as described above, it is configured such that the apparatus further includes the display unit 4 for displaying the image output by the image output unit 64 (control unit 6) and that the image output unit 64 is configured to display the X-ray image 10 and the identification image 40 on the display unit 4.

With this configuration, an operator, such as, e.g., a doctor, can easily compare the portion of the foreign object 200 colored in the identification image 40 with the position (region) of the X-ray image 10 corresponding to the portion of the colored foreign object 200 by visually recognizing both the X-ray image 10 and the identification image 40 displayed on the display unit 4. Therefore, an operator, such as, e.g., a doctor, can easily recognize the portion identified as the foreign object 200 in the X-ray image 10.

Further, in this embodiment, as described above, the image output unit 64 (control unit 6) is configured to cause the display unit 4 to display the X-ray image 10 with the identification image 40 superimposed thereon.

With this configuration, the identification image 40 in which the portion identified as the foreign object 200 has been colored is superimposed on the X-ray image 10. Therefore, unlike the case in which the X-ray image 10 and the identification image 40 are displayed side by side, the region identified as the foreign object 200 in the X-ray image 10 can be confirmed without moving the viewpoint between the X-ray image 10 and the identification image 40. As a result, an operator, such as, e.g., a doctor, can more easily recognize the portion of the X-ray image 10 identified as the foreign object 200 in the X-ray image.

[Effects of Image Processing Method by this Embodiment]

In the image processing method according to the X-ray imaging apparatus 100 of this embodiment, the following effects can be obtained.

In the image processing method of this embodiment is configured as described above to generate an enhanced image 30 in which the foreign object 200 included in X-ray image 10 is enhanced based on the enhanced image 10 and the removed image 20 in which the foreign object 200 included in X-ray image 10 has been removed from the X-ray image 10 by the trained model 51 generated by machine learning. Based on the generated enhanced image 30, an identification image 40 for identifying the foreign object 200 is generated by coloring the portion corresponding to the enhanced foreign object 200.

This allows the foreign object 200 to be enhanced without enhancing the structure, such as, e.g., the bone of the subject 101, by extracting the foreign object 200 based on the X-ray image 10 including the foreign object 200 and a removed image 20 in which the foreign object 200 has been removed, unlike the case in which both the structure, such as, e.g., the bone of the subject 101 and the foreign object 200 included in the subject 101 are enhanced by the foreign object enhancement processing, such as, e.g., edge detection processing. Therefore, in the case of generating the identification image 40 for identifying the foreign object 200 based on the enhanced image 30 in which the foreign object 200 is enhanced, the foreign object 200 in the identification image 40 can be effectively extracted and colored. Thus, the colored foreign object 200 can be easily identified by visually recognizing the effectively colored identification image 40 corresponding to the enhanced foreign object 200. As a result, it is possible to provide an image processing method capable of easily identifying the foreign object 200 included in the X-ray image 10 in the case of confirming the foreign object 200 left behind in the body of the subject 101 by generating the enhanced image 30.

[Effects of Generation Method of Trained Model by this Embodiment]

In the generation method of a trained model according to this embodiment, the following effects can be acquired.

In the generation method of the trained model according to this embodiment, as described above, in order to generate the identification image 40 for identifying the foreign object 200 by coloring the portion corresponding to the foreign object 200, the trained model 51 for outputting the removed image 20 in which the foreign object 200 included in the X-ray image 10 has been removed from the X-ray image 10 is generated, based on the training input X-ray image 310 and the training output removed image 320.

With this, the removed image 20 in which the foreign object 200 has been removed can be generated based on the trained model 51 generated by machine learning, and therefore, the foreign object 200 in the X-ray image 10 can be extracted, based on the X-ray image 10 including the foreign object 200 and the removed image 20 in which the foreign object 200 has been removed. Accordingly, it is possible to generate the enhanced image 30 in which the foreign object 200 has been enhanced while suppressing the enhancement to the structure, such as, e.g., the bone of the subject 101. Therefore, the identification image 40 for identifying the foreign object 200 can be easily generated by coloring the portion corresponding to the enhanced foreign object 200. As a result, the portion corresponding to the colored foreign object 200 can be easily identified by visually recognizing the identification image 40. Therefore, it is possible to provide a generation method of the trained model capable of easily identifying the foreign object 200 included in the X-ray image 10.

### Modified Embodiment

It should be understood that the embodiments disclosed here are examples in all respects and are not restrictive. The scope of the present invention is shown by claims rather than the descriptions of the embodiments described above, and includes all changes (modifications) within the meaning of equivalent to the claims.

That is, in the above-described embodiment, an example is shown in which it is configured such that the identification image generation unit 63 (control unit 6) generates the identification image 40 by identifying the linear structure of the foreign object 200 from the enhanced image 30 and coloring based on the identified linear structure, but the present invention is not limited thereto. For example, the identification image 40 may be generated by coloring based on the pixel value of the enhanced image 30 rather than the pattern recognition of the linear structure (shapes). In other words, the identification image 40 may be colored based on the concentration of the pixel value in a predetermined region of the enhanced image 30.

In the above-described embodiment, an example is shown in which it is configured such that the identification image generation unit 63 (control unit 6) generates the identification image 40 as a heat map image colored so as to change according to the density in a predetermined region including the site in which the linear structure of the foreign object 200 is arranged, but the present invention is not limited thereto. For example, it may be configured such that a threshold is given to the density of the linear structure without changing color to produce the identification image 40 in which a region greater than the threshold is colored with one color.

In the above-described embodiment, an example is shown in which it is configured such that the removed image generation unit 61 (control unit 6) divides the X-ray image 10 into a plurality of regions and generates the removed image 20 from the X-ray image 10 in a state in which it is divided into a plurality of regions, based on the trained model 51 generated by machine learning, but the present invention is not limited thereto. For example, it may be configured such that the removed image 20 is generated from one X-ray image 10 without dividing the X-ray image 10.

In the above-described embodiment, an example is shown in which the enhanced image generation unit 62 (control unit 6) generates the enhanced image 30 in which the foreign object 200 included in the X-ray image 10 has been enhanced by acquiring (subtracting processing) the difference between the X-ray image 10 and the removed image 20, but the present invention is not limited thereto. For example, it may be configured such that the enhanced image 30 is generated from the X-ray image not by performing the subtracting processing but by performing dividing processing for dividing the removed image 20 by the X-ray image 10.

Further, in the above-described embodiment, an example is shown in which the foreign object 200 includes a surgical operation gauze, a suture needle, and a forceps, but the present invention is not limited thereto. For example, the foreign object 200 may include a bolt, a fastening clip, and the like.

Further, in the above-described embodiment, an example is shown in which the apparatus further includes the display unit 4 for displaying the images output by the image output unit 64 (control unit 6), but the present invention is not limited thereto. For example, it may be configured such that the X-ray image 10 and the image, such as, e.g., the identification image 40 output by the image output unit 64 are displayed on a display device, such as, e.g., an external monitor, provided separately from the X-ray imaging apparatus 100.

Further, in the above-described embodiment, an example is shown in which the image output unit 64 (control unit 6) superimposes the identification image 40 on the X-ray image 10 and display it on the display unit 4, but the present invention is not limited thereto. For example, it may be configured such that the X-ray image 10 and the identification image 40 are displayed side by side on the display unit 4.

In the above-described embodiment, an example is shown in which the identification image 40 is generated so that the density (detected value) of the linear structure (shape) can be identified by the difference in brightness with the red color as a reference, but the present invention is not limited thereto. For example, it may be configured such that the identification image 40 is generated such that the distribution of the detected values of the linear shape can be identified by changing the hue. That is, when the detection value is large, red may be displayed, and the change of the detection value may be represented in the order of red, yellow, green, and blue.

In the above-described embodiment, an example is shown in which the control processing for the generation of the X-ray image 10 and the control processing for the generation of the identification image 40 are respectively performed by the X-ray image generation unit 3 and the control unit 6 configured as separate hardware, but the present invention is not limited thereto. For example, it may be configured such that the X-ray image 10 and the identification image 40 are generated by a common control unit (hardware).

In the above-described embodiment, an example is shown in which the removed image generation unit 61, the enhanced image generation unit 62, the identification image generation unit 63, and the image output unit 64 are configured as functional blocks (software) in one hardware (control unit 6), but the present invention is not limited thereto. For example, each of the removed image generation unit 61, the enhanced image generation unit 62, the identification image generation unit 63, and the image output unit 64 may be configured by separate hardware.

In the above-described embodiment, an example is shown in which the trained model 51 is generated by the learning device 300 separated from the X-ray imaging apparatus 100, but the present invention is not limited thereto. For example, the trained model 51 may be generated by the X-ray imaging apparatus 100.

Further, in the above-described embodiment, an example is shown in which the trained model 51 is generated based on a U-Net, which is one type of a fully convolution network (Fully Convolution Network: FCN), but the present invention is not limited thereto. For example, the trained model 51 may be generated based on a CNN (Convolutional Neural Network) including a fully connected layer. Further, the trained model 51 may be generated based on an Encoder-Decoder model other than a U-Net, such as, e.g., a SegNet and a PSPNet.

### [Aspects]

It will be understood by those skilled in the art that the above-described exemplary embodiments are concrete examples of the following aspects.

### (Item 1)

An X-ray imaging apparatus for performing X-ray imaging to identify a foreign object left behind in a body of a subject after a surgical operation, comprising:

an X-ray irradiation unit configured to irradiate the subject with X-rays;

an X-ray detection unit configured to detect X-rays emitted from the X-ray irradiation unit;

an X-ray image generation unit configured to generate an X-ray image based on a detection signal of X-rays detected by the X-ray detection unit; and

a control unit,

wherein the control unit includes:

an enhanced image generation unit configured to generate an enhanced image in which a foreign object included in an X-ray image has been enhanced, based on the X-ray image and a removed image in which the foreign object included in the X-ray image has been removed;

an identification image generation unit configured to generate an identification image for identifying the foreign object by coloring a portion corresponding to the enhanced foreign object, based on the enhanced image generated by the enhanced image generation unit; and

an image output unit configured to output the identification image generated by the identification image generation unit.

### (Item 2)

The X-ray imaging apparatus as recited in claim 1

wherein the identification image generation unit is configured to generate the identification image by identifying a linear structure of the foreign object from the enhanced image and coloring based on the identified linear structure.

### (Item 3)

The X-ray imaging apparatus as recited in the above-described Item 2,

wherein the identification image generation unit is configured to acquire a density in a predetermined region including a site in which the linear structure of the foreign object identified from the enhanced image is arranged and generate the identification image as a heat map image colored so as to change according to the density.

### (Item 4)

The X-ray imaging apparatus as recited in the above-described Item 2 or 3,

wherein the control unit further includes a removed image generation unit for generating the removed image in which the foreign object included in the X-ray image has been removed from the X-ray image, based on the trained model generated by machine learning,

wherein the removed image generation unit is configured to divide the X-ray image into a plurality of regions and generate the removed image from the X-ray image in a state in which the X-ray image is divided into the plurality of regions, based on the trained model generated by machine learning, and

wherein the identification image generation unit is configured to generate the identification image by identifying the linear structure of the foreign object from the enhanced image generated based on the X-ray image and the removed image generated from the X-ray image in a state in which the X-ray image is divided into a plurality of regions and coloring based on the identified linear structure.

### (Item 5)

The X-ray imaging apparatus as recited in any one of the above-described Items 2 to 4

wherein the enhanced image generation unit is configured to generate the enhanced image in which the foreign object included in the X-ray image has been enhanced by acquiring a difference between the X-ray image and the removed image, and

wherein the identification image generation unit is configured to generate the identification image by identifying the linear structure of the foreign object from the enhanced image generated by acquiring the difference between the removed image and the X-ray image and coloring based on the identified linear structure.

### (Item 6)

The X-ray imaging apparatus as recited in any one of the above-described Items 2 to 5,

wherein the foreign object includes a surgical operation gauze, a suture needle, and a forceps, and

wherein the identification image generation unit is configured to generate the identification image by identifying a linear structure corresponding to the surgical operation gauze, the suture needle, or the forceps from the enhanced image generated by the enhanced image generation unit and coloring based on the identified linear structure.

### (Item 7)

The X-ray imaging apparatus as recited in any one of the above-described Items 1 to 6, further comprising:

a display unit configured to display an image output by the image output unit,

wherein the image output unit is configured to cause the X-ray display unit to display the X-ray image and the identification image.

### (Item 8)

The X-ray imaging apparatus as recited in the above-described Item 7 wherein the image output unit is configured to cause the display unit to display the X-ray image with the identification image superimposed on the X-ray image.

### (Item 9)

An image processing method, comprising the steps of:

irradiating a subject with X-rays to identify a foreign object left behind in a body of a subject after a surgical operation;

detecting the X-rays;

generating an X-ray image based on a detection signal of the detected X-rays;

generating an enhanced image in which the foreign object included in the X-ray image has been enhanced, based on the X-ray image and a removed image in which the foreign object included in the X-ray image has been removed from the X-ray image by a trained model generated by machine learning;

generating an identification image for identifying the foreign object by coloring a portion corresponding to the enhanced foreign object, based on the generated enhanced image; and

outputting the generated identification image.

### (Item 10)

A trained model generation method comprising the steps of:

acquire a training input X-ray image generated to simulate an X-ray image generated by irradiating a subject after a surgical operation with a foreign object left behind in the body with X-rays;

acquiring a training output removed image in which the foreign object has been removed from the training input X-ray image; and

generating a trained model for outputting a removed image in which the foreign object included in the X-ray image has been removed from the X-ray image by machine learning, based on the training input X-ray image and a training output removed image, in order to generate an identification image for identifying the foreign object by coloring a portion corresponding to the foreign object.

1. An X-ray imaging apparatus for performing X-ray imaging to identify a foreign object left behind in a body of a subject after a surgical operation, the X-ray imaging apparatus comprising:
an X-ray irradiation unit configured to irradiate the subject with X-rays;
an X-ray detection unit configured to detect X-rays emitted from the X-ray irradiation unit;
an X-ray image generation unit configured to generate an X-ray image based on a detection signal of X-rays detected by the X-ray detection unit; and
a control unit,
wherein the control unit includes:
an enhanced image generation unit configured to generate an enhanced image in which a foreign object included in an X-ray image has been enhanced, based on the X-ray image and a removed image in which the foreign object included in the X-ray image has been removed;
an identification image generation unit configured to generate an identification image for identifying the foreign object by coloring a portion corresponding to the enhanced foreign object, based on the enhanced image generated by the enhanced image generation unit; and
an image output unit configured to output the identification image generated by the identification image generation unit.

2. The X-ray imaging apparatus as recited in claim 1,
wherein the identification image generation unit is configured to generate the identification image by identifying a linear structure of the foreign object from the enhanced image and coloring based on the identified linear structure.

3. The X-ray imaging apparatus as recited in claim 2,
wherein the identification image generation unit is configured to acquire a density in a predetermined region including a site in which the linear structure of the foreign object identified from the enhanced image is arranged and generate the identification image as a heat map image colored so as to change according to the density.

4. The X-ray imaging apparatus as recited in claim 2,
wherein the control unit further includes a removed image generation unit for generating the removed image in which the foreign object included in the X-ray image has been removed from the X-ray image, based on the trained model generated by machine learning,
wherein the removed image generation unit is configured to divide the X-ray image into a plurality of regions and generate the removed image from the X-ray image in a state in which the X-ray image is divided into the plurality of regions, based on the trained model generated by machine learning, and
wherein the identification image generation unit is configured to generate the identification image by identifying the linear structure of the foreign object from the enhanced image generated based on the X-ray image and the removed image generated from the X-ray image in a state in which the X-ray image is divided into a plurality of regions and coloring based on the identified linear structure.

5. The X-ray imaging apparatus as recited in claim 2,
wherein the enhanced image generation unit is configured to generate the enhanced image in which the foreign object included in the X-ray image has been enhanced by acquiring a difference between the X-ray image and the removed image, and
wherein the identification image generation unit is configured to generate the identification image by identifying the linear structure of the foreign object from the enhanced image generated by acquiring the difference between the removed image and the X-ray image and coloring based on the identified linear structure.

6. The X-ray imaging apparatus as recited in claim 2,
wherein the foreign object includes a surgical operation gauze, a suture needle, and a forceps, and
wherein the identification image generation unit is configured to generate the identification image by identifying a linear structure corresponding to the surgical operation gauze, the suture needle, or the forceps from the enhanced image generated by the enhanced image generation unit and coloring based on the identified linear structure.

7. The X-ray imaging apparatus as recited in claim 1, further comprising:
a display unit configured to display an image output by the image output unit,
wherein the image output unit is configured to cause the X-ray display unit to display the X-ray image and the identification image.

8. The X-ray imaging apparatus as recited in claim 7,
wherein the image output unit is configured to cause the display unit to display the X-ray image with the identification image superimposed on the X-ray image.

9. An image processing method, comprising the steps of:
irradiating a subject with X-rays to identify a foreign object left behind in a body of a subject after a surgical operation;
detecting the X-rays;
generating an X-ray image based on a detection signal of the detected X-rays;
generating an enhanced image in which the foreign object included in the X-ray image has been enhanced, based on the X-ray image and a removed image in which the foreign object included in the X-ray image has been removed from the X-ray image by a trained model generated by machine learning;
generating an identification image for identifying the foreign object by coloring a portion corresponding to the enhanced foreign object, based on the generated enhanced image; and
outputting the generated identification image.

10. A trained model generation method comprising the steps of:
acquiring a training input X-ray image generated to simulate an X-ray image generated by irradiating a subject after a surgical operation in which a foreign object is left behind in the body with X-rays;
acquiring a training output removed image in which the foreign object has been removed from the training input X-ray image; and
generating a trained model for outputting a removed image in which the foreign object included in the X-ray image has been removed from the X-ray image by machine learning, based on the training input X-ray image and the training output removed image, in order to generate an identification image for identifying the foreign object by coloring a portion corresponding to the foreign object.
